(19) United States
(12) Patent Application Publication
(10) Pub. No.: US 2023/0180950 A1
(43) Pub. Date: Jun. 15, 2023

(54) SIDE-SLEEPER PILLOW FOR USE WITH CONTINUOUS POSITIVE AIRWAY PRESSURE SYSTEMS AND DEVICES

(71) Applicant: CVB INC, Logan, UT (US)

(72) Inventors: Joshua Fenton, Nibley, UT (US); Jeffrey Giles, Hyrum, UT (US)

(21) Appl. No.: 17/546,894

(22) Filed: Dec. 9, 2021

(51) Int. Cl. A47G 9/10; A47G 9/00

(52) U.S. Cl. CPC A47G 9/10; A47G 9/007; A61M 16/0003

(57) ABSTRACT

A pillow comprising a first component having a head-supporting material with an upper surface, a lower surface, and a raised wall formed between the upper and lower surfaces, wherein the wall includes a truncated first wall portion with a beveled upper edge and a beveled lower edge; a second wall portion adjacent to the first wall portion; a truncated third wall portion adjacent to the second wall portion and opposite the first wall portion with a beveled upper edge and a beveled lower edge; a fourth wall portion adjacent to the third wall portion and opposite the second wall portion having a cutout region configured to receive the shoulder of a user; and a removable second component having the shape of the first pillow component and being configured to enclose the first component, wherein the second component includes a tether configured to secure a length of tubing.

### BACKGROUND

The disclosed inventive subject matter relates in general to supportive devices used for sleep such as head pillows and the like, and more specifically to a contoured head pillow for use by side-sleepers who also use various masks and tubes associated with continuous positive airway pressure systems and devices.

Sleep apnea is a common sleep disorder in which repeated pauses in breathing or periods of shallow breathing occur during sleep. Each pause can last for a few seconds to a few minutes and may happen many times each night. Loud snoring may also occur and choking or snorting may happen as breathing resumes. Because the disorder disrupts normal sleep, those affected by this disorder may experience sleepiness or daytime tiredness. Obstructive sleep apnea (OSA) is the most common category of sleep-disordered breathing. During OSA, the muscle tone of the body relaxes during sleep and the walls of soft tissue found at the level of the throat in the human airway collapse, thereby obstructing normal breathing. Chronic severe obstructive sleep apnea requires treatment to prevent low blood oxygen (hypoxemia), sleep deprivation, and other potentially dangerous complications such as decreased cognitive functioning, cerebrovascular diseases (e.g., hypertension, coronary artery disease, and stroke), and diabetes.

Continuous positive airway pressure (CPAP) is a form of positive airway pressure ventilation wherein a constant level of air pressure which is greater than atmospheric pressure is continuously applied to the upper respiratory tract of a person. The application of this positive pressure is intended to prevent upper airway collapse such as that which occurs during obstructive sleep apnea. CPAP is the most effective treatment for moderate to severe obstructive sleep apnea and has been shown to be 100% effective at eliminating obstructive sleep apnea in the majority of people who use the therapy per the recommendations of their doctor.

Despite the demonstrated effectiveness of CPAP systems, devices, and methods, a significant problem associated with the use of CPAP systems is patient compliance. Because CPAP systems require the use of plastic tubing and face masks that must be worn throughout the night by the patient, many people find CPAP treatment to be uncomfortable, inconvenient, or otherwise undesirable. Additionally, the use of a regular or standard pillow with a CPAP face mask may result in general discomfort to the wearer of the CPAP mask and may even result in the mask becoming displaced during sleep, which can repeatedly break the required seal between the mask and the wearer's face. This is especially true if the wearer regularly sleeps on his or her side. Accordingly, there is an ongoing need for a supportive head pillow that can be used with CPAP systems in a manner than will not negatively affect the use and effectiveness of such systems.

### SUMMARY

The following provides a summary of certain example implementations of the disclosed inventive subject matter. This summary is not an extensive overview and is not intended to identify key or critical aspects or elements of the disclosed inventive subject matter or to delineate its scope. However, it is to be understood that the use of indefinite articles in the language used to describe and claim the disclosed inventive subject matter is not intended in any way to limit the described inventive subject matter. Rather the use of “a” or “an” should be interpreted to mean “at least one” or “one or more”.

One implementation of the disclosed article provides a first supportive head pillow configured for use with a continuous positive airway pressure face mask, comprising a first pillow component, wherein the first pillow component comprises at least one piece of head-supporting material having an upper surface, a lower surface, and a raised wall formed between the upper and lower surfaces, and wherein the wall includes a truncated first wall portion, wherein the truncated first wall portion further includes a beveled upper edge and a beveled lower edge; a second wall portion adjacent to the first wall portion; a truncated third wall portion adjacent to the second wall portion and opposite the first wall portion, wherein the truncated third wall portion further includes a beveled upper edge and a beveled lower edge; and a fourth wall portion adjacent to the third wall portion and opposite the second wall portion, wherein the fourth wall portion includes a cutout region configured to receive the shoulder of a user.

The first pillow component may include at least one antimicrobial material infused therein. The second wall portion may be outwardly rounded. The at least piece of head-supporting material may include a plurality of ventilation apertures formed therein and passing either partially or completely therethrough. The head-supporting material may include polyurethane memory foam core. The second pillow component may have the general shape of the first pillow component, may be configured to enclose the first pillow component, and may include a tether configured to secure a length of tubing. The second pillow component may include a coating of at least one antimicrobial material, may include a breathable fabric, and may include a blend of materials such as, for example, polyester and nylon or other materials.

Another implementation of the disclosed article provides a second supportive head pillow configured for use with a continuous positive airway pressure face mask, comprising a first pillow component, wherein the first pillow component comprises at least one piece of head-supporting foam core material having an upper surface, a lower surface, and a raised wall formed between the upper and lower surfaces, and wherein the wall includes a truncated first wall portion; a second wall portion adjacent to the first wall portion; a truncated third wall portion adjacent to the second wall portion and opposite the first wall portion; a fourth wall portion adjacent to the third wall portion and opposite the second wall portion, wherein the fourth wall portion includes a cutout region configured to receive the shoulder of a user; and a removable second pillow component, wherein the second pillow component has the general shape of the first pillow component and is configured to enclose the first pillow component.

The at least one piece of head-supporting material may include a plurality of ventilation apertures formed therein and passing either partially or completely therethrough. The truncated first wall portion may further includes a beveled upper edge and a beveled lower edge, and the truncated third wall portion may further include a beveled upper edge and a beveled lower edge. The second wall portion may be outwardly curved or rounded. The second pillow component may include a tether configured to secure a length of tubing. The first pillow component may include at least one antimicrobial material infused therein, and the second pillow component may include a coating of at least one antimicrobial material. The second pillow component may include a breathable fabric that includes a blend of polyester and nylon or other materials.

Still another implementation of the disclosed article provides a third supportive head pillow configured for use with a continuous positive airway pressure facemask, comprising a first pillow component, wherein the first pillow component comprises at least one piece of head-supporting material having an upper surface, a lower surface, and a raised wall formed between the upper and lower surfaces, and wherein the wall includes a truncated first wall portion, wherein the truncated first wall portion further includes a beveled upper edge and a beveled lower edge; a second wall portion adjacent to the first wall portion wherein the second wall portion is outwardly curved; a truncated third wall portion adjacent to the second wall portion and opposite the first wall portion, wherein the truncated third wall portion further includes a beveled upper edge and a beveled lower edge; a fourth wall portion adjacent to the third wall portion and opposite the second wall portion, wherein the fourth wall portion includes a cutout region configured to receive the shoulder of a user; and a removable second pillow component, wherein the second pillow component wherein the second pillow component has the shape of the first pillow component and is configured to enclose the first pillow component, and wherein the second pillow component includes a tether configured to secure a length of tubing.

The at least one piece of head-supporting material may include polyurethane memory foam core having a plurality of ventilation apertures formed therein and passing either partially or completely therethrough. The first pillow component may include at least one antimicrobial material infused therein and the second pillow component may include a coating of at least one antimicrobial material. The second pillow component may include a breathable fabric and the breathable fabric may include polyester and nylon or other materials.

It should be appreciated that all combinations of the foregoing concepts and additional concepts discussed in greater detail below (provided such concepts are not mutually inconsistent) are contemplated as being part of the inventive subject matter disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more example implementations of the disclosed inventive subject matter and, together with the general description given above and detailed description given below, serve to explain the principles of the disclosed subject matter, and wherein:

FIG. 1 is a front perspective view of an example implementation of a supportive head pillow configured for use with a continuous positive airway pressure face mask in accordance with the disclosed article;

FIG. 2 is a top view of the supportive head pillow of FIG. 1;

FIG. 3 is front view of the supportive head pillow of FIG. 1 showing the shoulder-receiving cutout region;

FIG. 4 is front perspective view of an example implementation of a cover for use with the supportive head pillow of FIG. 1;

FIG. 5 is a top view of the cover of FIG. 4;

FIG. 6 is a front view of the cover of FIG. 4;

FIG. 7 is a front view depicting a patient or user of the pillow of FIG. 1 properly positioned on the pillow, wherein, for the sake of comparison, the broken lines indicate the location or position of the body of a standard pillow; and

FIG. 8 is a top view depicting the patient or user of the pillow of FIG. 1 as shown in FIG. 7, wherein, for the sake of comparison, the broken lines indicate the location or position of the side edges of a standard pillow.

### DETAILED DESCRIPTION

Example implementations are now described with reference to the Figures. Reference numerals are used throughout the detailed description to refer to the various elements and structures. Although the following detailed description contains many specifics for the purposes of illustration, a person of ordinary skill in the art will appreciate that many variations and alterations to the following details are within the scope of the disclosed inventive subject matter. Accordingly, the following implementations are set forth without any loss of generality to, and without imposing limitations upon, the claimed subject matter.

The disclosed technology includes various implementations of a head support or pillow for use with positive airway pressure systems and devices. The disclosed contoured pillow is particularly useful for people who are side-sleepers and who experience discomfort or inconvenience when using standard or traditional pillows that interfere with the proper placement of masking and tubing used with positive airway pressure systems and devices.

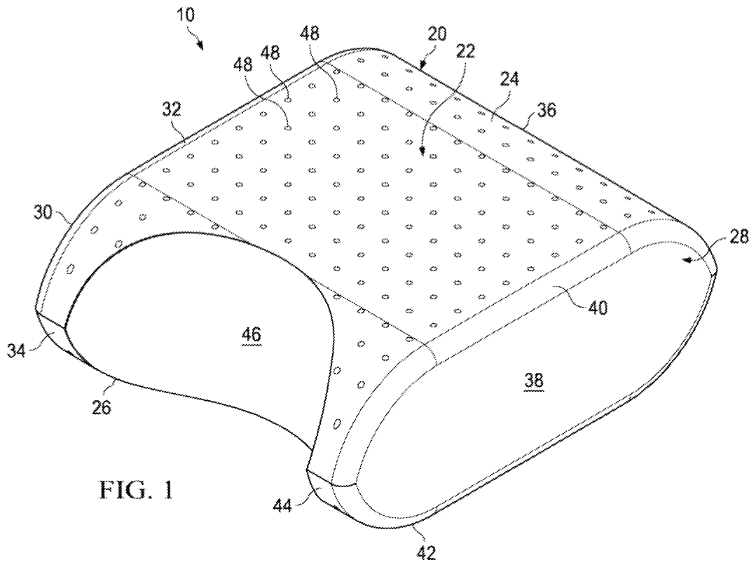

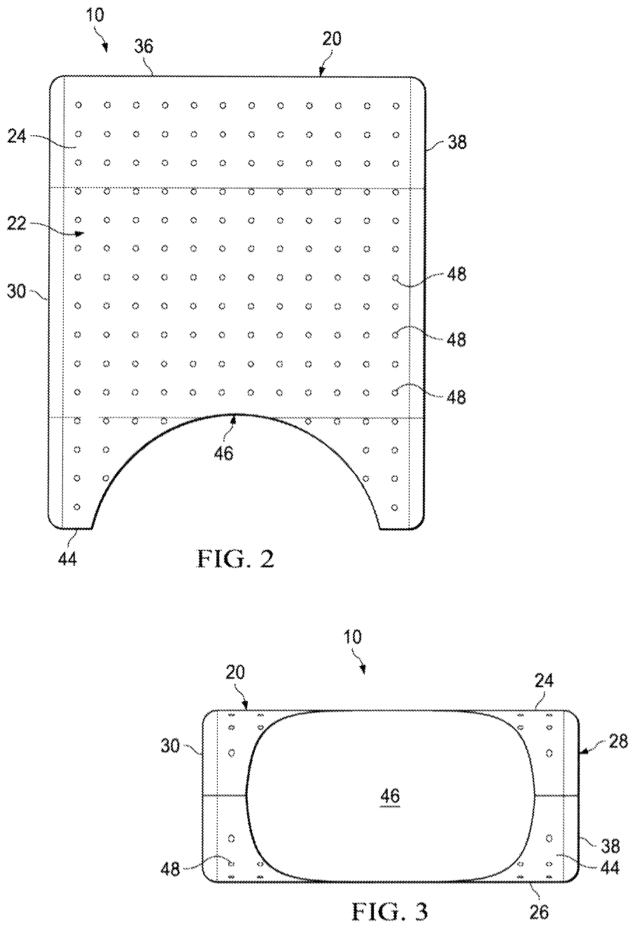

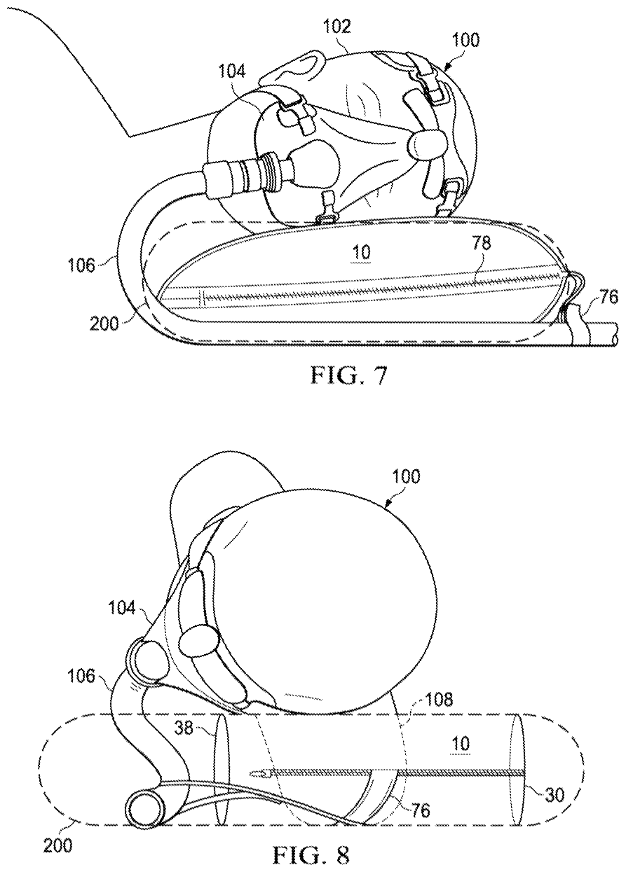

FIG. 1 provides a front perspective view of an example implementation of a supportive head pillow configured for use with a continuous positive airway pressure (CPAP) face mask. FIG. 2 provides a top view of the supportive head pillow of FIG. 1 and FIG. 3 provides a front view of the supportive head pillow of FIG. 1 showing the shoulder-receiving cutout region. As shown in FIGS. 1-3, pillow 10 includes first pillow component 20, which is typically made from at least one piece of supportive material 22. First pillow component 20 is generally square or rectangular in shape and includes upper surface 24 and lower surface 26 which are separated by a wall 28, which has a predetermined height or thickness. Wall 28 includes truncated first wall portion 30 having a beveled upper edge 32 and beveled lower edge 34, second wall portion 36 which may be outwardly rounded, truncated third wall portion 38 having beveled upper edge 40 and beveled lower edge 42, and fourth wall portion 44 having cutout region 46, which is configured to receive the shoulder of patient or user 100 (see FIGS. 7-8).

Supportive material 22 may include premium polyurethane memory foam core or similar materials and may include a plurality of stamped ventilation holes or apertures 48 formed therein. The density of the premium polyurethane memory foam core is optimized to accommodate a wide range of user sizes and weights. In some implementations, the foam includes antimicrobial properties from bamboo charcoal or other biocide or biocide infused therein. The stamped cooling or ventilation holes allow for air movement and are spaced at a predetermined distance from one another (e.g., one inch (2.54 cm)) to provide ideal compression of the memory foam and allow alignment of the user's head and spinal column.

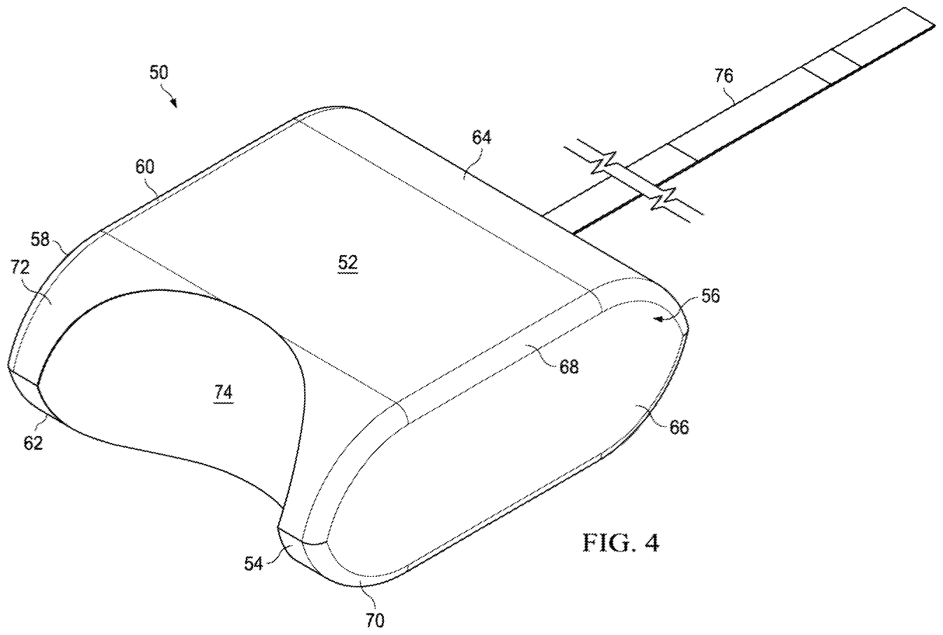

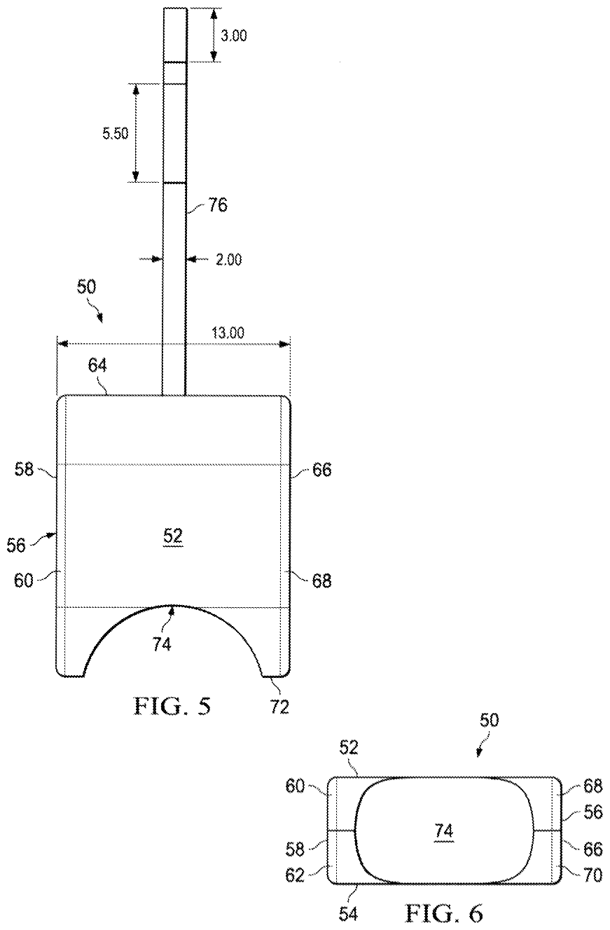

FIG. 4 provides a front perspective view of an example implementation of a cover for use with the supportive head pillow of FIG. 1. FIG. 5 provides a top view of the cover of FIG. 4 and FIG. 6 provides a front view of the cover of FIG. 4. Cover or second pillow component 50 generally matches the shape and contours of first pillow component 20 includes upper surface 52, and lower surface 54 and are separated by a wall 56, which has a predetermined height or thickness. Wall 56 includes truncated (i.e., shortened or brought inward relative to the outermost edge of a standard pillow configuration) first wall portion 58 having beveled upper edge 60 and beveled lower edge 62, second wall portion 64, truncated third wall portion 66 having beveled upper edge 68 and beveled lower edge 70, and fourth wall portion 72 having cutout region 74, which is configured to receive the shoulder of patient or user 100 (see FIGS. 7-8). Strap or tether 76 (which may be attached to first pillow component 20 in some implementations) is typically attached to second wall portion 64 and extends outward therefrom for securing or immobilizing CPAP tubing. Tether 76 may be of any useful length. A zipper 78 may be provided for ease of removing second pillow component 50 from first pillow component 20.

In some embodiments, cover or second pillow component 50 may include a blend of polyester and open nylon or tight nylon. This fabric blend permits moisture wicking and breathability which provides increased air flow and durability. Cover or second pillow component 50 may be treated with an antimicrobial application that inhibits the growth of bacteria and other harmful substances.

FIG. 7 provides a front view depicting a patient or user of the pillow of FIG. 1 properly positioned on the pillow, wherein, for the sake of comparison, the broken lines indicate the location or position of the body of a standard pillow 200; and FIG. 8 provides a top view depicting a patient or user of the pillow of FIG. 1 as shown in FIG. 7, wherein, for the sake of comparison, the broken lines indicate the location or position of the side edges of a standard pillow 200. The design of pillow 10 facilitates preservation of the required seal between CPAP mask 104 and face 102 of patient/user 100. The truncated sides and beveled edges of pillow 10 reduce the overall length of the pillow compared to a standard pillow and provide the clearance necessary for pillow 10 to not touch or interfere with CPAP mask 104 or CPAP tubing 106, during sleep. The beveled edges are positioned to ensure that whether a side or stomach sleeper uses a full-face, nasal pillow, or nose CPAP mask, the pillow will create no pressure, thereby giving the CPAP mask zero interference and allowing the mask to sit completely above sleeping surface. Shoulder cutout regions (46 and 74) allow shoulder 108 to fit into a recess in pillow 10 which permits the head of patient/user 100 to rest in a position that aligns the head, neck, and back for optimal sleeping when patient/user 100 is lying on their side. Tether 76 secures CPAP tubing 106 to prevent or reduce any undesirable pulling on CPAP mask 104 that may occur with movement and that make break the necessary seal. Pillow 10 may also be used by back or stomach sleepers by simply rotating the pillow 180 degrees.

In an example implementation, truncated first wall portion 30 and truncated third wall portion 38 are 15.75 inches (40.00 cm) in length; second wall portion 36 and fourth wall portion 44 are 13.00 inches (33.02 cm) in length; cutout region 46 is 10.00 inches (25.40 cm) in length; wall 28 is 6.00 inches (15.24 cm) in height; and tether 76 is 22.00 inches (55.88 cm) in length. Numerous other dimensions and configurations are possible.

All literature and similar material cited in this application, including, but not limited to, patents, patent applications, articles, books, treatises, and web pages, regardless of the format of such literature and similar materials, are expressly incorporated by reference in their entirety. Should one or more of the incorporated references and similar materials differs from or contradicts this application, including but not limited to defined terms, term usage, described techniques, or the like, this application controls.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about”, if or when used throughout this specification describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

There may be many alternate ways to implement the disclosed inventive subject matter. Various functions and elements described herein may be partitioned differently from those shown without departing from the scope of the disclosed inventive subject matter. Generic principles defined herein may be applied to other implementations. Different numbers of a given module or unit may be employed, a different type or types of a given module or unit may be employed, a given module or unit may be added, or a given module or unit may be omitted.

Regarding this disclosure, the term “a plurality of” refers to two or more than two. Unless otherwise clearly defined, orientation or positional relations indicated by terms such as “upper” and “lower” are based on the orientation or positional relations as shown in the figures, only for facilitating description of the present invention and simplifying the description, rather than indicating or implying that the referred devices or elements must be in a particular orientation or constructed or operated in the particular orientation, and therefore they should not be construed as limiting the present invention. The terms “connected”, “mounted”, “fixed”, etc. should be understood in a broad sense. For example, “connected” may be a fixed connection, a detachable connection, or an integral connection; a direct connection, or an indirect connection through an intermediate medium. For an ordinary skilled in the art, the specific meaning of the above terms in the present invention may be understood according to specific circumstances.

It should be appreciated that all combinations of the foregoing concepts and additional concepts discussed in greater detail herein (provided such concepts are not mutually inconsistent) are contemplated as being part of the disclosed inventive subject matter. In particular, all combinations of claimed subject matter appearing at the end of this disclosure are contemplated as being part of the inventive subject matter disclosed herein. While the disclosed inventive subject matter has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed inventive subject matter in its broader aspects is not limited to any of the specific details, representative devices and methods, and/or illustrative examples shown and described. Accordingly, departures may be made from such details without departing from the spirit or scope of the general inventive concept.

What is claimed:

1. A supportive head pillow configured for use with a continuous positive airway pressure face mask, comprising:
(a) a first pillow component, wherein the first pillow component comprises at least one piece of head-supporting material having an upper surface, a lower surface, and a raised wall formed between the upper and lower surfaces, and wherein the wall includes:
(i) a truncated first wall portion, wherein the truncated first wall portion further includes a beveled upper edge and a beveled lower edge;
(ii) a second wall portion adjacent to the first wall portion;
(iii) a truncated third wall portion adjacent to the second wall portion and opposite the first wall portion, wherein the truncated third wall portion further includes a beveled upper edge and a beveled lower edge; and
(iv) a fourth wall portion adjacent to the third wall portion and opposite the second wall portion, wherein the fourth wall portion includes a cutout region configured to receive the shoulder of a user.

2. The supportive head pillow of claim 1, wherein the first pillow component includes at least one antimicrobial material infused therein.

3. The supportive head pillow of claim 1, wherein the second wall portion is outwardly rounded.

4. The supportive head pillow of claim 1, wherein the at least piece of head-supporting material includes a plurality of ventilation apertures formed therein and passing either partially or completely therethrough.

5. The supportive head pillow of claim 1, wherein the at least piece of head-supporting material includes polyurethane memory foam core.

6. The supportive head pillow of claim 1, further comprising a second pillow component, wherein the second pillow component has the shape of the first pillow component and is configured to enclose the first pillow component, and wherein the second pillow component includes a tether configured to secure a length of tubing.

7. The supportive head pillow of claim 6, wherein the second pillow component includes a coating of at least one antimicrobial material.

8. The supportive head pillow of claim 6, wherein the second pillow component includes a breathable fabric.

9. The supportive head pillow of claim 6, wherein the second pillow component includes a blend of materials, and wherein the materials include polyester and nylon.

10. A supportive head pillow configured for use with a continuous positive airway pressure face mask, comprising:
(a) a first pillow component, wherein the first pillow component comprises at least one piece of head-supporting foam core material having an upper surface, a lower surface, and a raised wall formed between the upper and lower surfaces, and wherein the wall includes:
(i) a truncated first wall portion;
(ii) a second wall portion adjacent to the first wall portion;
(iii) a truncated third wall portion adjacent to the second wall portion and opposite the first wall portion;
(iv) a fourth wall portion adjacent to the third wall portion and opposite the second wall portion, wherein the fourth wall portion includes a cutout region configured to receive the shoulder of a user; and
(b) a removable second pillow component, wherein the second pillow component has the shape of the first pillow component and is configured to enclose the first pillow component.

11. The supportive head pillow of claim 10, wherein the at least one piece of head-supporting material includes a plurality of ventilation apertures formed therein and passing either partially or completely therethrough.

12. The supportive head pillow of claim 10, wherein the truncated first wall portion further includes a beveled upper edge and a beveled lower edge, and wherein the truncated third wall portion further includes a beveled upper edge and a beveled lower edge.

13. The supportive head pillow of claim 10, wherein the second wall portion is outwardly curved or rounded.

14. The supportive head pillow of claim 10, wherein the second pillow component includes a tether configured to secure a length of tubing.

15. The supportive head pillow of claim 10, wherein the first pillow component includes at least one antimicrobial material infused therein, and wherein the second pillow component includes a coating of at least one antimicrobial material.

16. The supportive head pillow of claim 10, wherein the second pillow component includes a breathable fabric that includes a blend of polyester and nylon.

17. A supportive head pillow configured for use with a continuous positive airway pressure face mask, comprising:
(a) a first pillow component, wherein the first pillow component comprises at least one piece of head-supporting material having an upper surface, a lower surface, and a raised wall formed between the upper and lower surfaces, and wherein the wall includes:
(i) a truncated first wall portion, wherein the truncated first wall portion further includes a beveled upper edge and a beveled lower edge;
(ii) a second wall portion adjacent to the first wall portion wherein the second wall portion is outwardly curved;
(iii) a truncated third wall portion adjacent to the second wall portion and opposite the first wall portion, wherein the truncated third wall portion further includes a beveled upper edge and a beveled lower edge;
(iv) a fourth wall portion adjacent to the third wall portion and opposite the second wall portion, wherein the fourth wall portion includes a cutout region configured to receive the shoulder of a user; and
(b) a removable second pillow component, wherein the second pillow component wherein the second pillow component has the shape of the first pillow component and is configured to enclose the first pillow component, and wherein the second pillow component includes a tether configured to secure a length of tubing.

18. The supportive head pillow of claim 17, wherein the at least one piece of head-supporting material includes polyurethane memory foam core having a plurality of ventilation apertures formed therein and passing either partially or completely therethrough.

19. The supportive head pillow of claim 17, wherein the first pillow component includes at least one antimicrobial material infused therein, and wherein the second pillow component includes a coating of at least one antimicrobial material.

20. The supportive head pillow of claim 17, wherein the second pillow component includes a breathable fabric, and wherein the breathable fabric includes polyester and nylon.
